(19) United States
(12) Patent Application Publication
(10) Pub. No.: US 2005/0013833 A1
(43) Pub. Date: Jan. 20, 2005

(54) Composition based on lipid lamellar vesicles incorporating at least a dhea compound

(76) Inventor: Jean-Thierry Simonnet, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/484,000

(22) PCT Filed: Jul. 18, 2002
(86) PCT No.: PCT/FR02/02573

(30) Foreign Application Priority Data
Jul. 27, 2001 (FR) 01 10111

(51) Int. Cl. A61K 35/78; A61K 9/14; A61K 31/57

(52) U.S. Cl. 424/400; 424/757; 424/773; 514/26; 514/177

(57) ABSTRACT

The present invention relates to a composition comprising: a dispersion, in an outer aqueous phase, of vesicles formed by lipid lamellar phases comprising at least one amphiphilic lipid and encapsulating an inner hydrophilic phase, the said lamellar phases not comprising succinic and/or hemisuccinic derivatives, and at least one DHEA-based compound included in the said lamellar phases. The incorporation of the DHEA-based compound into the lamellar phases of vesicles makes it possible to avoid its recrystallization in the outer aqueous phase and to improve its bioavailability.

The invention relates to a composition comprising vesicles formed from lipid lamellar phases containing at least one DHEA-based compound. The invention also relates to a process for preparing this composition.

For the purposes of the invention, the term “DHEA-based compound” means DHEA itself, DHEA precursors or DHEA derivatives.

Many patents exist describing cosmetic or dermatological compositions for topical application comprising dehydroepiandrosterone and/or its derivatives. For example, patent U.S. Pat. No. 5,989,568 describes the use of dehydroepiandrosterone sulphate in a topical composition for treating wrinkles and fine lines and/or for combating cutaneous and/or subcutaneous slackening and/or for reviving the radiance of the skin.

DHEA, or dehydroepiandrosterone, is a natural steroid produced essentially by the adrenal glands. Exogenous DHEA, administered topically or orally, is known for its capacity to promote keratinization of the epidermis (JP-07 196 467) and to treat dry skin by increasing the endogenous production and the secretion of sebum, thus reinforcing the skin's barrier effect (U.S. Pat. No. 4,496,556). The use of DHEA for overcoming dermal atrophy by inhibiting the loss of collagen and of connective tissue has also been described in patent U.S. Pat. No. 5,843,932. Finally, the Applicant has demonstrated the capacity of DHEA to combat the weathered appearance of the skin (FR 00/00349) and to modulate the pigmentation of the skin and the hair (FR 99/12773). These properties of DHEA make it a candidate of choice as an anti-ageing active agent.

Among the DHEA metabolites, particular attention has been paid in recent years to 7α-hydroxy DHEA. Specifically, it has been demonstrated that this metabolite, which does not have the hormonal activity of DHEA, makes it possible to increase the proliferation of fibroblasts and the viability of human keratinocytes, and has free-radical-scavenging effects (WO 98/40074). It has also been demonstrated on rats (WO 00/28996) that 7α-hydroxy DHEA increases the thickness of the dermis and the elastin and collagen content of the skin. It has thus been suggested to use this DHEA metabolite to prevent and/or treat the harmful effects of UV on the skin, to combat wrinkles and to increase the firmness and tonicity of the skin.

However, DHEA-based compounds have the drawback of being very sparingly soluble in cosmetic solvents, and of crystallizing in the presence of an aqueous phase. This results in a more or less pronounced loss of efficacy of these compositions depending on the degree of crystallization, which is counter to the desired objective.

Furthermore, DHEA-based compounds have better bioavailability in the skin when they are in dissolved form in cosmetic supports, and furthermore at high levels, than when they are in crystalline form with a poorly controlled crystal size.

For the purposes of the patent application, the term “bioavailability” means the molecular penetration of the active agent under consideration into the live layers of the skin and in particular of the epidermis. It will be sought to enable the penetrated concentration to be as high as possible, so as to increase the amount of active agent arriving as far as the live layers of the skin.

It is possible to dissolve DHEA-based compounds at 25° C. in certain solvents such as propylene glycol, liposoluble screening agents or 2-alkyl alkanols, for example, but, to do this, it is necessary to have very high concentrations of solvents in order to dissolve large amounts of DHEA-based compounds. Now, since these solvents are preferably oily, it will rather be sought to limit their content in the final composition in order to have the most acceptable cosmetic feel possible and also to limit any problems of tolerance.

There is thus still a need to dissolve DHEA-based compounds in a physiologically acceptable vehicle.

The Applicant has now discovered that DHEA and/or its derivatives and/or its precursors can be introduced into compositions as constituents of lipid lamellar phases of vesicular type encapsulating a hydrophilic phase. These vesicles may be either niosomes of the type described in patent applications EP-958 856, EP-582 503, EP-455 528 or EP-43327, or liposomes of standard type. DHEA and/or its derivatives and/or precursors become, in this type of structure, one of the constituents of the lamellar phases.

It is known to those skilled in the art that cholesterol can be used to reinforce the leaktightness of the wall of such vesicles. The Applicant has now found, surprisingly, that it is possible, in the constitution of the lipid lamellar phases, to replace cholesterol with at least one DHEA-based compound.

These compounds, although having a structure similar to cholesterol, are more polar: they are characterized by solubility parameters revealing a polarity of the DHEA-based compounds that is higher than that of cholesterol.

For the purposes of the invention, the term “solubility parameters” means the Hansen solubility parameters δ_{d}, δₚ and δₕ. These parameters are defined in the document S. Paint Technology 30, 195 (1967) “The Three Dimensional Solubility Parameter—Key to Paint Component Affinities”.

The solubility parameters of cholesterol, DHEA and its analogues are presented in Table 1 below.

TABLE 1

| | Cholesterol | DHEA | 7-Keto-DHEA | 7a-OH-DHEA |
|---|---|---|---|---|
| δₐ* | 7.31 | 9.95 | 10.72 | 13.21 |

*δₐ = [(δₚ)² + (δₕ)²]^{1/2}

Given these differences in polarity, the pure and simple replacement of cholesterol with one of these molecules was not obvious. Specifically, these polarities imply a higher affinity with water than in the case of cholesterol and induce higher Ostwald maturation sensitivity (Kabalnov et al., J. Colloid and Interface Sci. 118 (1987) 590-597), which should therefore lead to rapid recrystallization of the DHEA-based compounds in the continuous phase of the compositions containing them.

The introduction of a DHEA-based compound is performed by replacing all or some of the cholesterol with the said DHEA-based compound. The latter compound is then intimately associated with the other lipids constituting the lamellar phase of the vesicles (hydrogenated or non-hydrogenated lecithin, nonionic surfactants capable of forming lamellar phases when they are combined with cholesterol, as described in patents EP 958 856, EP 582 503, EP 455 528 and EP 43327, and ionic surfactants intended to stabilize the vesicles obtained). They do not recrystallize in the continuous medium which is water and in which they are insoluble.

Admittedly, a person skilled in the art knows the formulation of liposomes based on a 5β-steroid.

Thus, international patent application WO-97/13500 describes liposomes comprising a lipid or a lipid compound and a 5β-steroid, DHEA or an organic acid derived from a 5β-steroid or DHEA. The vesicles described in the said patent application are based on an α-tocopheryl hemisuccinate salt prepared beforehand, and also on a Tris salt of cholesteryl hemisuccinate, which is also prepared beforehand. These vesicles are especially intended for treating obesity and/or diabetes and/or hypercorticoidism. The preparation of these vesicles is long and complex and does not allow large-scale exploitation.

One subject of the present invention is thus a composition comprising:
a dispersion, in an outer aqueous phase, of vesicles formed by lipid lamellar phases comprising at least one amphiphilic lipid and encapsulating an inner hydrophilic phase and dispersed in an outer aqueous phase, the said lamellar phases not comprising succinic and/or hemisuccinic derivatives, and
at least one DHEA-based compound included in the said lamellar phases.

The incorporation of the said DHEA-based compound into the lamellar phases of the vesicles allows it to be made available in a dissolved form and to efficiently avoid its crystallization in the aqueous phase. In addition, the vesicles according to the invention promote the bioavailability of the said DHEA-based compound in the skin. Finally, the gradual degradation of the vesicles according to the invention allows a controlled release of the said DHEA-based compound that they contain.

For the purposes of the patent application, the term “dissolved form” means a dispersion in molecular form in a lyotropic phase of liquid crystal type, of lamellar or hexagonal type, no crystallization of the active agent being visible to the naked eye or by cross-polarized optical microscopy.

The DHEA-based compounds that may be used according to the invention are chosen from DHEA itself, DHEA precursors and DHEA derivatives.

As DHEA precursors that may be used according to the invention, mention may be made of its biological precursors that may be converted into DHEA during metabolism, and also its chemical precursors that may be converted into DHEA by exogenous chemical reaction. Examples of biological precursors are Δ5-pregnenolone, 17α-hydroxypregnenolone and 17α-hydroxypregnenolone sulphate, this list not intended to be limiting. Examples of chemical precursors are sapogenins and derivatives thereof such as diosgenin (or 5-spirostene-3β-ol), hecogenin, hecogenin acetate, smilagenin and sarsapogenin, tigogenin, yamogenin and yuccagenin, and also natural extracts containing them, in particular fenugreek and extracts of Dioscorea plants such as extract of wild yam, this list not intended to be limiting.

AS DHEA derivative that may be used according to the invention, mention may be made of both its metabolic derivatives and its chemical derivatives.

Metabolic derivatives that may especially be mentioned include A5-androstene-3,17-diol and Δ4-androstene-3,17-dione, and also 7α-OH DHEA, 7β-OH DHEA, 11α-OH DHEA and 7-keto-DHEA, this list not intended to be limiting. 7α-OH DHEA is preferred for use in the present invention. A process for preparing this compound is described in particular in patent applications FR 2 771 105 and WO 94/08588.

Chemical derivatives that may especially be mentioned include DHEA salts and in particular water-soluble salts such as DHEA sulphate. Mention may also be made of esters such as hydroxycarboxylic acid esters of DHEA, described in particular in U.S. Pat. No. 2,736,537 or other esters such as DHEA salicylate, DHEA acetate, DHEA valerate (or n-heptanoate) and DHEA enanthate. Mention may also be made of DHEA derivatives (DHEA carbamates, DHEA 2-hydroxymalonate esters and DHEA amino acid esters) described in patent application FR 00/03846 in the name of the Applicant. 3-Alkyl esters of 7-keto-DHEA, for example 3-acetoxy-7-keto-DHEA, may also be mentioned.

Other chemical DHEA derivatives that are suitable for use in the present invention are the derivatives of formula (1): 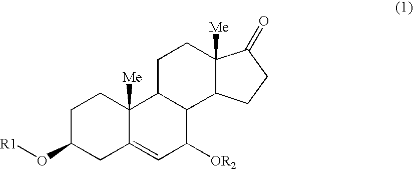
in which:
R₁ and R₂ are chosen independently from:
   a saturated or unsaturated, linear, branched or cyclic C₁-C₁₂ alkyl group optionally containing one or more hetero atoms, and optionally substituted with one or more groups chosen from —OR′ and/or —SR′ and/or —COOR′ and/or —NR′R′ and/or halogen and/or sulphate and/or phosphate and/or aryl and/or heterocycle, the said heterocycle advantageously being chosen from an indole, a pyrimidine, a piperidine, a morpholine, a pyran, a furan, a piperazine and a pyridine;
   an alkylcarbonyl group, the C₁-C₂₄ alkyl portion of which is saturated or unsaturated, linear, branched or cyclic, and optionally substituted with one or more groups chosen from —OR′ and/or —SR′ and/or —COOR′ and/or —NR′R′ and/or halogen and/or sulphate and/or phosphate and/or aryl and/or heterocycle, the said heterocycle advantageously being chosen from an indole, a pyrimidine, a piperidine, a morpholine, a pyran, a furan, a piperazine and a pyridine;
   an arylcarbonyl group, preferably a phenylcarbonyl group, or an arylalkylcarbonyl group, preferably a benzylcarbonyl group, optionally substituted with one or more groups —OR′ and/or —SR′ and/or —COOR′ and/or —NR′R′ and/or halogen and/or aryl and/or heterocycle;
   a group O═P(OH)OR′;
   a group (O)₂SOR′;
   a trialkylsilyl group (SiR′₃) in which the 3 groups R′ may be identical or different;
   a carbonyloxyalkyl group (R′OCO);
   a carbonylaminoalkyl group (R′NHCO); in which R′ is chosen from a hydrogen atom, a saturated or unsaturated, linear, branched or cyclic C₁-C₁₂ and preferably C₁-C₆ alkyl group optionally containing one or more hetero atoms, optionally functionalized with one or more groups —OR″, —COOR″, halogen, —NR″R″; or with an aryl group, preferably a phenyl group, optionally functionalized with one or more groups —OR″, —COOR″, halogen or —NR″R″;
R″ representing a hydrogen atom or a saturated or unsaturated, linear, branched or cyclic alkyl chain, preferably of C₁-C₆,
it being understood that, in each of the groups —NR′R′ and —NR″R″, the substituents R′ or R″, respectively, are identical or different.

Among the derivatives of formula (1) that may be mentioned in particular are 7-OH-DHEA diesters and more preferably 3-O-acetyl-7-benzoyloxydihydroepiandro-sterone, which is especially available from the company Gattefosse under the trade name 3-acetoxy-7-benzoate DHEA.

The DHEA-based compound(s) may represent from 0.1% to 50% by weight and preferably from 1% to 25% by weight of the lipid composition constituting the membrane of the vesicles, i.e. the lipid lamellar phases.

The vesicles according to the invention are preferably formed by, or comprise, from 1 to 25 leaflets of substantially concentric lamellar phases of bimolecular type.

These leaflets are obtained from lipids that have both the property of forming mesomorphic phases, the state of organization of which is intermediate between the crystalline state and the liquid state, and of swelling in the presence of an aqueous solution to form the said lamellar phases which will give, with stirring, the vesicles dispersed in the aqueous phase.

The vesicles according to the invention are lipid lamellar vesicles with an aqueous core, i.e. encapsulating a hydrophilic phase, which is the inner hydrophilic phase. These vesicles may be either niosomes of the type described in patent application EP 0 582 503, the teaching of which is incorporated herein by reference, or the like, or liposomes of standard type.

In the case of niosomes, the lamellar phases comprise at least one nonionic amphiphilic lipid, chosen from optionally oxyethylenated alkyl or polyalkyl esters of polyol, and optionally oxyethylenated polyol ethers, with a melting point of at least 40° C.

Nonionic amphiphilic lipids that are suitable for use in the present invention are especially glycolipids of natural or synthetic origin (for example cerebrosides), or mixtures of polyol esters of at least one acid with a saturated hydrocarbon-based chain containing at least 14 carbon atoms, and also polyol ethers of at least one alcohol with a saturated hydrocarbon-based chain containing at least 14 carbon atoms.

The term “mixtures of esters” means not only mixtures of pure esters of different chemical families, but also any product containing several chemically pure polyol esters of the same family in variable proportions, such as polyglycerol esters comprising a random number of glycerol units.

The nonionic amphiphilic lipid may thus consist of a mixture of esters of at least one polyol chosen from the group formed by polyethylene glycol comprising from 1 to 60 ethylene oxide units, sorbitan, sorbitan bearing 2 to 60 ethylene oxide units, glycerol bearing 2 to 30 ethylene oxide units, polyglycerols comprising 2 to 15 glycerol units, sucroses, glucoses bearing 2 to 30 ethylene oxide units, and at least one fatty acid comprising a saturated or unsaturated, linear or branched C₁₄-C₂₀ hydrocarbon-based chain.

As polyol ethers that may be used according to the invention, mention may be made of:
linear or branched polyglycerol ethers having the respective formulae
   R—(OCH₂—CH(OH)—CH₂)n-OH (I)
   and
   R—(O—CH₂—CH(CH₂OH))n-OH (II)
   in which n is an integer between 1 and 6, preferably equal to 2, and R is a radical chosen from:
(a) a linear or branched, saturated or unsaturated aliphatic chain containing from 14 to 30 carbon atoms, such as a tetradecyl or hexadecyl radical or the alkyl radical of oleyl alcohol or of isostearyl alcohol;
(b) a hydrocarbon-based radical of lanolin alcohol;
(c) a 2-hydroxyalkyl residue of a (-diol, the hydrocarbon-based chain of which contains at least 14 carbon atoms; and
polyoxyethylenated fatty alcohols, such as oleyl alcohol oxyethylenated with 10 mol (“Brij 96” product sold by the company ICI Atlas).

Moreover, with the aim of improving the stability of the niosomes as described above, the lamellar phases may also comprise an ionic amphiphilic lipid. This lipid may be chosen from anionic lipids and cationic lipids.

The anionic amphiphilic lipids that are suitable for use in the invention may be:
neutralized anionic lipids, preferably chosen from alkali metal salts of dicetyl phosphate and of dimyristyl phosphate, in particular the sodium and potassium salts, alkali metal salts of phosphatidic acid, in particular the sodium salt, the alkali metal salts of cholesteryl sulphate, in particular the sodium salt, alkali metal salts of cholesteryl phosphate, in particular the sodium salt, salts of lipoamino acids such as monosodium and disodium acylglutamates, more particularly the disodium salt of N-stearoyl-L-glutamic acid sold under the name Acylglutamate HS21 by the company Ajinomoto,
amphoteric lipids, preferably phospholipids, in particular pure soybean phosphatidylethanolamine;
alkylsulphonic derivatives, in particular the compounds of formula: 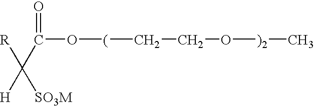
   in which R represents a C₁₂ to C₂₂ hydrocarbon-based radical, in particular the C₁₆H₃₃ and C₁₈H₃₇ radicals, and M is an alkali metal, preferably sodium.

The cationic amphiphilic lipids that may be used in the vesicles of the invention as ionic amphiphilic lipids may be chosen more particularly from the group formed by quaternary ammonium salts and fatty amines and salts thereof.

Among the ammonium salts that are particularly suitable for use in the invention, mention will be made of: 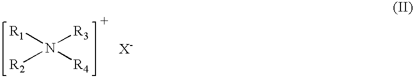
those represented by the general formula (II) below: in which the radicals R₁ to R₄, which may be identical or different, represent a linear or branched aliphatic radical containing from 1 to 30 carbon atoms, or an aromatic radical such as aryl or alkylaryl. The aliphatic radicals can comprise hetero atoms such as, in particular, oxygen, nitrogen, sulphur or halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl radicals containing from 1 to 30 carbon atoms approximately; X is an anion chosen from the group formed by halides, phosphates, acetates, lactates, (C₂-C₆)alkylsulphates and alkyl or alkylarylsulphonates. Quaternary ammonium salts of formula (II) which are preferred, on the one hand, are tetraalkylammonium chlorides such as, for example, dialkyldimethylammonium or alkyltrimethylammonium chlorides, in which the alkyl radical contains from 12 to 22 carbon atoms approximately, in particular behenyltrimethylammonium chloride, distearyldimethylammonium chloride, cetyltrimethylammonium chloride or benzyldimethylstearylammonium chloride, or alternatively, on the other hand, stearamidopropyl-dimethyl(myristyl acetate)ammonium chloride sold under the name Ceraphyl 70 by the company Van Dyk.
quaternary ammonium salts of imidazolinium, for example represented by the formula (III) below: 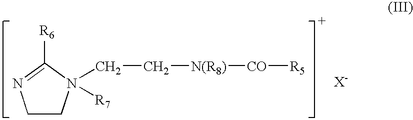
in which R₅ represents an alkenyl or alkyl radical containing from 8 to 30 carbon atoms, for example a tallow fatty acid derivative; R₆ represents a hydrogen atom, an alkyl radical containing from 1 to 4 carbon atoms or an alkenyl or alkyl radical containing from 8 to 30 carbon atoms; R₇ represents an alkyl radical containing from 1 to 4 carbon atoms; R₈ represents a hydrogen atom or an alkyl radical containing from 1 to 4 carbon atoms; X is an anion chosen from the group formed by halides, phosphates, acetates, lactates, alkyl sulphates and alkyl or alkylaryl sulphonates. Preferably, R₅ and R₆ denote a mixture of alkenyl or alkyl radicals containing from 12 to 21 carbon atoms, for example tallow fatty acid derivatives, R₇ denotes a methyl radical and R₈ denotes hydrogen. Such a product is sold, for example, under the name Rewoquat W75 by the company Rewo.
diquaternary ammonium salts represented by the formula (IV) below: 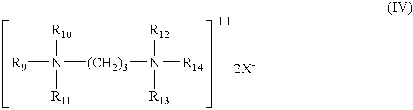
in which R₆ denotes an aliphatic radical containing from 16 to 30 carbon atoms approximately; R₇, R₈, R₉, R₁₀ and R₁₁, which may be identical or different, are chosen from hydrogen or an alkyl radical containing from 1 to 4 carbon atoms; and X is an anion chosen from the group formed by halides, acetates, phosphates, nitrates and methyl sulphates. Such diquaternary ammonium salts in particular comprise propane tallow diammonium dichloride.

Besides the nonionic and ionic amphiphilic lipids, the lamellar phases of the vesicles of niosomal type may also contain at least one additive chosen from sterols, fatty-chain alcohols and diols, fatty-chain amines and quaternary ammonium derivatives thereof.

It is preferred to use cholesterol, which, besides its cosmetic and/or dermopharmaceutical activity associated with its capacity to reconstitute skin lipids, makes it possible to improve the stability of the vesicles by avoiding the crystallization of the surfactants with which it is combined. By improving the impermeability of the lamellar phases of the vesicles, cholesterol also makes it possible to increase the power of retention of the water-soluble active agents possibly contained in the hydrophilic phase encapsulated by the niosomes.

The lamellar phases of the vesicles of niosomal type may contain, for example, from 35% to 90% by weight of nonionic amphiphilic lipid, from 0 to 20% by weight of ionic amphiphilic lipid, from 5% to 50% by weight of cholesterol and from 0.1% to 50% by weight of at least one DHEA-based compound relative to the total weight of the lipids constituting the lamellar phase.

As mentioned previously, the lipid lamellar vesicles according to the invention may comprise not only vesicles of nonionic type such as niosomes, but also standard liposomes, comprising at least one ionic amphiphilic lipid such as a natural or synthetic phospholipid, in particular lecithin, which is preferably hydrogenated, combined either with cholesterol and optionally with an ionic surfactant, or with an oxyethylenated phytosterol comprising from 2 to 50 ethylene oxide units.

In this embodiment variant of the invention, the vesicles may comprise from 50% to 99% by weight of lecithin, from 50% to 1% by weight of a mixture of cholesterol and of at least one DHEA-based compound, and from 0 to 20% by weight of ionic surfactant, relative to the total weight of the lipids constituting the lamellar phase. As a variant, they may comprise from 40% to 80% by weight of lecithin and from 20% to 60% by weight of a mixture of oxyethylenated phytosterol and of at least one DHEA-based compound, relative to the total weight of the lipids constituting the lamellar phase. In both cases, the amount of DHEA-based compound generally represents from 0.1% to 50% of the total weight of the lipids constituting the lamellar phase.

The methods for manufacturing the vesicles according to the invention are known to those skilled in the art, but the preferred methods are the following:

Bangham Method

The vesicular lipids are dissolved in a mixture of organic solvents. This mixture is then placed in a round-bottomed flask and the solvents are evaporated off on a rotary evaporator under reduced pressure. A lipid film then forms. After total evaporation of the solvents, the film is hydrated with an aqueous solution, with vigorous stirring. The temperature is adjusted to the melting point of the lipids. A suspension of liposomes is then obtained. It is then possible to homogenize it using ultrasound.

Method via Direct Hydration of the Lipids

The lipids may or may not have been preassociated (via melting or via solvent). The lipid mixture is then introduced with vigorous stirring (for example a rotor-stator) into an aqueous solution at an adjusted temperature. After a few minutes (generally from 5 to 90 minutes), a suspension of liposomes is obtained. What has thus been made is an aqueous dispersion of DHEA (and/or of its derivatives and/or its precursors), which does not recrystallize over time (at least three days at 25° C.), since it is intimately associated with the components of the vesicular membrane.

Irrespective of the invention embodiment used (niosomes or liposomes), the lipids constituting the vesicles usually represent from 1% to 20% and preferably from 1% to 10% of the total weight of the composition.

In the composition according to the invention, the vesicles with an aqueous core described above (niosomes or liposomes) are dispersed in an aqueous dispersion phase, or outer aqueous phase, comprising a physiologically acceptable medium, i.e. a medium that is compatible with the skin or its integuments, and possibly with mucous and/or semi-mucous membranes.

The aqueous dispersion phase may be gelled. Examples of gelling agents that may be used according to the invention include carboxyvinyl polymers (carbomer), acrylic copolymers such as acrylate/alkylacrylate copolymers, polyacrylamides such as partially neutralized and highly crosslinked polyacrylamidomethylpropanesulphonic acid, polysaccharides, natural gums and clays.

As a variant, the aqueous dispersion phase may comprise an oily phase dispersed in the said aqueous phase (oil-in-water emulsion) and/or may itself be dispersed in an oily phase (water-in-oil emulsion).

As oils that may be used according to the invention, mention may be made of animal or plant oils, natural or synthetic essential oils, hydrocarbons such as isohexadecane and liquid paraffin, halocarbons and silicone oils.

As animal or plant oils that may be used according to the invention, mention may be made especially of animal or plant oils formed from fatty acid esters of polyols, in particular liquid triglycerides, for example sunflower oil, maize oil, soybean oil, marrow oil, grapeseed oil, jojoba oil, sesame seed oil, hazelnut oil, fish oils, glyceryl tricaprocaprylate or plant or animal oils of formula R₁COOR₂, in which formula R₁ represents a higher fatty acid residue containing from 7 to 19 carbon atoms and R₂ represents a branched hydrocarbon-based chain containing from 3 to 20 carbon atoms, for example purcellin oil.

As essential oils that may be used according to the invention, mention may be made of natural or synthetic essential oils, for instance eucalyptus oil, lavandin oil, lavender oil, vetiver oil, Litsea cubeba oil, lemon oil, sandalwood oil, rosemary oil, camomile oil, savory oil, walnut oil, nutmeg oil, cinnamon oil, hyssop oil, caraway oil, orange oil, geraniol oil, cade oil and bergamot oil.

As halocarbons that may be used according to the invention, mention may be made of fluorocarbons such as fluoroamines, for example perfluorotributylamine, fluorohydrocarbons, for example perfluorodecahydronaphthalene, fluoroesters and fluoroethers.

When the vesicles according to the invention are dispersed in the aqueous phase of an oil-in-water emulsion, the said emulsion may comprise surfactants other than those constituting the vesicles, provided that these surfactants do not dissolve the vesicles, forming micelles.

However, according to another possibility, when the composition according to the invention is in the form of an oil-in-water emulsion, it cannot contain any surfactant other than those forming the lipid lamellar vesicles. Specifically, the vesicles according to the invention may be capable of stabilizing a dispersion of oil droplets in the aqueous dispersion phase, without it being necessary to add a surfactant to the said aqueous phase.

Needless to say, as a variant, the composition according to the invention may be in the form of a water-in-oil-in-water or oil-in-water-in-oil multiple emulsion in which the aqueous and oily phases are as defined above.

The vesicles of the compositions according to the invention may contain, in a known manner, one or more active compound(s) with cosmetic and/or dermopharmaceutical activity, which, depending on their solubility characteristics, may be in different locations.

If the active agents are water-soluble, they are introduced into the encapsulated hydrophilic phase of the vesicles.

If the active agents are liposoluble, they are introduced into the lipid phase constituting the membrane.

If the active agents are amphiphilic, they are distributed between the lipid phase and the encapsulated hydrophilic phase, with a partition coefficient that varies depending on the nature of the amphiphilic active agent and the respective compositions of the lipid phase and of the encapsulated hydrophilic phase.

In a known manner, the composition according to the invention may also contain adjuvants that are common in cosmetics, such as preserving agents, antioxidants, solvents, fragrances, odour absorbers, neutralizers, sunscreens, polymers, emulsifiers and co-emulsifiers, and dyestuffs.

Active agents that may especially be used include depigmenting agents, emollients, moisturizers, antiseborrhoeic agents, anti-acne agents, agents for promoting regrowth of the hair, keratolytic and/or desquamating agents, anti-wrinkle and tensioning agents, and vitamins, and mixtures thereof.

The compositions according to the invention may also contain at least one UV-screening agent (or sunscreen), which may be a chemical screening agent or a physical blocking agent or a mixture of such screening agents.

The amounts of the various constituents of the composition according to the invention are those conventionally used in cosmetics.

Needless to say, a person skilled in the art will take care to select the optional additional additives and/or the amount thereof such that the advantageous properties of the composition according to the invention are not, or are not substantially, adversely affected by the envisaged addition. In particular, these compounds must not harm the advantageous properties of the DHEA-based compound(s) that may be used according to the invention, or promote its (their) recrystallization.

The composition according to the invention may especially constitute protective/care/makeup products for the face, the body or the scalp and haircare products.

The present invention also relates to the cosmetic use of the composition mentioned above, for preventing or treating the signs of intrinsic or photo-induced ageing of the skin.

The invention also relates to the use of the composition mentioned above to manufacture a preparation for preventing or treating atrophy of the skin or of mucous membranes.

The invention will now be illustrated with the aid of the non-limiting examples that follow.

### EXAMPLES

### Example 1

### DHEA-Based Niosomes

| | |
|---|---|
| Sorbitan palmitate | 4% |
| Cholesterol | 4% |
| Vitamin E acetate | 0.5% |
| DHEA | 0.5% |
| Disodium acylglutamate | 1% |
| Distilled water | qs. 100% |

Procedure

These vesicles were prepared via the Bangham method.

The vesicles obtained have a mean size of less than 500 nm and show no recrystallization of the DHEA. They may be introduced into an emulsified support or simply gelled with a hydrophilic polymer of Carbomer or AMPS type.

### Example 2

### Composition Based on Niosomes Containing DHEA

| | |
|---|---|
| Diglyceryl distearate | 4.5% |
| Cholesterol | 4% |
| DHEA | 1% |
| Monosodium acylglutamate | 1% |
| Distilled water | qs. 60% |
| Oily phase | |
| Capric/caprylic triglyceride | 15% |
| Volatile silicone | 10% |
| Gelling phase | |
| Carbomer | 0.3% |
| Preserving agents | 1% |
| Distilled water | qs. 100% |
| Triethanolamine | 0.3% |

The lipid combination is prepared by mixing the constituents under hot conditions. This mixture, cooled to room temperature, is introduced into the aqueous phase at 90° C. Very vigorous stirring is maintained for 30 to 60 minutes. The suspension is then cooled to 60° C. and homogenized using a high-pressure homogenizer at 500 bar. A suspension of niosomes comprising DHEA, and with a mean size of less than 300 nm, is obtained.

The oily phase is introduced with vigorous stirring into this vesicular suspension, which has been cooled to a temperature of 30° C. This pre-emulsion is homogenized at 500 bar and the whole mixture, once cooled to 25° C., is then gelled with the gelling phase, which is dispersed using a deflocculator. A shiny, smooth, white cream containing 1% DHEA is obtained.

1. A composition comprising a dispersion of an outer aqueous phase, an inner hydrophilic phase and at least one DHEA compound,
wherein the outer aqueous phase comprises vesicles of lipid lamellar phases comprising at least one amphiphilic lipid,
wherein the lipid lamellar phases encapsulate the inner hydrophilic phase,
wherein the lamellar phases do not comprise a succinic derivative, or a hemisuccinic derivative or both, and
wherein the at least one DHEA compound is present in the lamellar phases.

2. The composition according to claim 1, wherein the DHEA compound is selected from the group consisting of DHEA, a DHEA precursor and a DHEA derivative.

3. The composition according to claim 2, comprising at least one of Δ5-pregnenolone, 17α-hydroxypregnenolone or 17 α-hydroxypregnenolone sulphate.

4. The composition according to claim 2, comprising at least one DHEA chemical precursor selected from the group consisting of a sapogenin, a natural extract of fenugreek and an extract of a Dioscorea plants

5. The composition according to claim 2, comprising at least one DHEA derivative selected from the group consisting of Δ5-androstene-3,17-diol, Δ4-androstene-3,17-dione, 7α-OH DHEA, 7β-OH DHEA, 11α-OH DHEA, 7-keto-DHEA, 3-acetoxy-7-keto-DHEA, DHEA sulphate, a hydroxycarboxylic acid ester of DHEA, DHEA salicylate, DHEA acetate, DHEA valerate, DHEA enanthate, a DHEA carbamate, a 2-hydroxymalonate ester of DHEA and an amino acid ester of DHEA.

6. The composition according to claim 2, comprising a DHEA derivative is represented by formula (1) 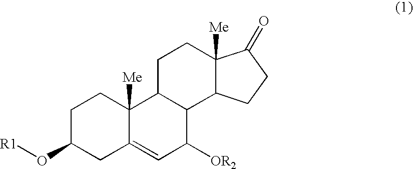
in which:
R₁ and R₂ are independently:
a saturated or unsaturated, linear, branched or cyclic C₁-C₁₂ alkyl group optionally containing one or more hetero atoms, and optionally substituted with one or more of an OR′, —SR6′, —COOR′, —NR′R′, halogen, sulphate, phosphate, aryl, or heterocycle group,
an alkylcarbonyl group, the C₁-C₂₄ alkyl portion of which is saturated or unsaturated, linear, branched or cyclic, and optionally substituted with one or more of a —OR′, —SR′, —COOR′, —NR′R, halogen, sulphate, phosphate, aryl, or heterocycle group,
an arylcarbonyl group, or an arylalkylcarbonyl group, optionally substituted with one or more groups of a —OR′, —SR′, —COOR′, —NR′R′, halogen, aryl or heterocycle group;
a group O═P(OH)OR′;
a group (O)₂SOR′;
a trialkylsilyl group of formula SiR′₃ in which the 3 groups R′ may be identical or different;
a carbonyloxyalkyl group of formula R′OCO;
a carbonylaminoalkyl group of formula R′NHCO;
in which R′ is a hydrogen atom, a saturated or unsaturated, linear, branched or cyclic C₁-C₁₂ group optionally containing one or more hetero atoms, optionally functionalized with one or more of a —OR″, —COOR″, halogen, —NR″R″, or aryl group, and optionally functionalized with one or more of a —OR″, —COOR″, halogen or —NR″R″ group;
R″ representing a hydrogen atom or a saturated or unsaturated, linear, branched or cyclic alkyl chain,
wherein each of the groups —NR′R′ and —NR″R″, the substituents R′ or R″, respectively, are identical or different.

7. The composition according to claim 6, comprising 3-O-acetyl-7-benzoyloxy-dehydroepiandrosterone.

8. The composition according to claim 1, wherein the DHEA compound represents from 0.1% to 50% by weight of the lipid lamellar phases.

9. The composition according to claim 1, wherein the lamellar phases comprise at least one nonionic amphiphilic lipid selected from the group consisting of an alkyl ester of a polyol, a polyalkyl ester of a polyol, an alkyl ether of a polyol, a polyalkyl ether of a polyol and oxyalkylerated compounds thereof, with a melting point of at least 40° C.

10. The composition according to claim 9, comprising a nonionic amphiphilic lipid comprising a mixture of polyol esters of at least one acid with a saturated hydrocarbon-based chain containing at least 14 carbon atoms.

11. The composition according to claim 9, comprising a nonionic amphiphilic lipid comprising a polyol ether of at least one alcohol with a saturated hydrocarbon-based chain containing at least 14 carbon atoms.

12. The composition according to claim 9, comprising at least one nonionic amphiphilic lipid consisting of a mixture of esters of at least one polyol selected from the group consisting of a polyethylene glycol comprising from 1 to 60 ethylene oxide units, sorbitan, sorbitan bearing 2 to 60 ethylene oxide units, glycerol bearing 2 to 30 ethylene oxide units, a polyglycerol comprising 2 to 15 glycerol units, a sucrose, a glucose bearing 2 to 30 ethylene oxide units, and a fatty acid comprising a saturated or unsaturated, linear or branched C₁₄-C₂₀ hydrocarbon-based chain.

13. The composition according to claim 1, wherein the lamellar phases comprise an ionic amphiphilic lipid.

14. The composition according to claim 13, wherein the ionic amphiphilic lipid is selected from the group consisting of an alkali metal salt of dicetyl, an alkali metal salt of dimyristyl phosphate; an alkali metal salt of cholesteryl sulphate; an alkali metal salt of cholesteryl phosphate; a monosodium acylglutamate; a disodium acylglutamate; a sodium salt of phosphatidic acid; a phospholipids; an acylgutamate, a alkylsulphonic derivative, and an ammonium salt represented by formula (II): 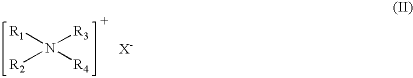
in which the radicals R₁ to R₄, which may be identical or different, represent a linear or branched aliphatic radical containing from 1 to 30 carbon atoms, an aromatic aryl radical, an aromatic radical, an alkylaryl radical; X is an anion selected from the group consisting of a halide, a phosphate, an acetate, a lactate, a (C₂-C₆)alkyl sulphate, an alkyl sulphonate and a alkylaryl sulphonate,
an quaternary ammonium salts of imidazolinium, represented, by formula (III): 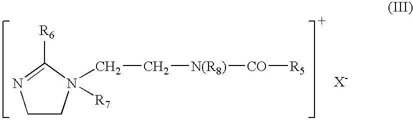
in which R₅ represents an alkenyl or alkyl radical containing from 8 to 30 carbon atoms, R₆ represents a hydrogen atom, an alkyl radical containing from 1 to 4 carbon atoms or an alkenyl or alkyl radical containing from 8 to 30 carbon atoms; R₇ represents an alkyl radical containing from 1 to 4 carbon atoms; R₈ represents a hydrogen atom or an alkyl radical containing from 1 to 4 carbon atoms; X is an anion selected from the group consisting of a halide, a phosphate, an acetate, a lactate, an alkyl sulphate, and an alkyl sulphonate and an alkylaryl sulphonate, and
diquaternary ammonium salts represented by formula (IV): 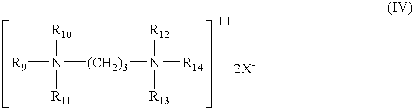
in which R₆ denotes an aliphatic radical containing from 16 to 30 carbon atoms approximately; R₇, R₈, R₉, R₁₀ and R₁₁, which may be identical or different, may be a hydrogen or an alkyl radical containing from 1 to 4 carbon atoms; and X is an anion selected from the group consisting of a halide, an acetate, a phosphate, a nitrate and a methyl sulphate.

15. The composition according to claim 9, wherein the lamellar phases contain comprise at least one additive selected from the group consisting of sterols, fatty-chain alcohols, fatty chain diols, fatty-chain amines and quaternary ammonium derivatives thereof.

16. The composition according to claim 15, comprising cholesterol.

17. The composition according to claim 16, wherein the lamellar phases comprise from 35% to 90% by weight of nonionic amphiphilic lipid, from 0 to 20% by weight of ionic amphiphilic lipid, from 5% to 50% by weight of cholesterol and from 0.1% to 50% by weight of the DHEA-compound relative to the total weight of the lipids comprising the lamellar phase.

18. The composition according to claim 1, wherein the lamellar phases comprise at least one phospholipids, combined either with cholesterol and optionally with an ionic surfactant, or with an oxyethylenated phytosterol comprising from 2 to 50 ethylene oxide units.

19. The composition according to claim 18, wherein the lamellar phase comprises lecithin.

20. The composition according to either claim 18, wherein the lipid vesicles comprise from 50% to 99% by weight of lecithin, from 50% to 1% by weight of a mixture of cholesterol and the DHEA compound, and from 0 to 20% by weight of one or more ionic surfactant relative to the total weight of the lipids comprising the lamellar phase.

21. The composition according to claim 18 wherein the lipid vesicles comprise from 40% to 80% by weight of lecithin and from 20% to 60% by weight of a mixture of oxyethylenated phytosterol and the DHEA compound relative to the total weight of the lipids comprising the lamellar phase.

22. The composition according to claim 1, wherein the lipids are in the vesicles and represent from 1% to 20% of the total weight of the composition.

23. The composition according to claim 1 in gel form.

24. The composition according to claim 1, further comprising an oily phase dispersed in the outer aqueous phase or a phase in which the outer aqueous phase is dispersed.

25. The composition according to claim 1, comprising at least one water-soluble, amphiphilic or liposoluble active agent.

26 (Canceled).

27 (Canceled).

28. The composition according to claim 4, comprising at least one sapogenin selected from the group consisting of diosgenin, hecogenin, smilagenin, sarsapogenin, tigogenin, yamogenin and yuccagenin.

29. The composition according to claim 6, wherein at least one of R₁ or R₂ is a group substituted with a heterocycle group selected from the group consisting of indole, a pyrimidine, a piperidine, a morpholine, a pyran, a furan, a piperazine and a pyridine.

30. The composition according to claim 1, wherein at least one of R₁ or R₂ is a phenylcarbonyl group.

31. The composition according to claim 6, wherein at least one of R₁ or R₂ is a benzylcarbonyl group.

32. The composition according to claim 6, wherein at least one of R₁ or R₂ is substituted with a C₁-C₆ alkyl group.

33. The composition according to claim 6, wherein at least one of R₁ or R₂ is substituted with a R′ group functionalized with a phenyl group.

34. The composition of claim 6, wherein at least one of R₁ or R₂ is substituted with a R′ group which is functionalized with a group containing a R″ group that is a C₁-C₆ alkyl chain.

35. The composition according to claim 1, comprising a DHEA compound present in an amount of from 1% to 25% by weight of the lipid lamellar phases.

36. The composition according to claim 18, wherein the lamellar phase comprises hydrogenated lecithin.

37. The composition according to claim 1, wherein the lipids are in vesicles and the lipids are present in an amount of from 1% to 10% of the total weight of the composition.

38. A cosmetic method comprising administering the composition of claim 1 to a mammal to prevent or treat the signs of at least one of intrinsic aging of the skin or photo-induced aging of the skin.

39. A composition comprising the composition of claim 1 and a physiologically or dermatologically acceptable diluent, excipient or carrier.
